(19) United States
(12) Patent Application Publication
(10) Pub. No.: US 2007/0015911 A1
(43) Pub. Date: Jan. 18, 2007

(54) Methods for biomarker discovery and diagnostic screening

(76) Inventors: Scott E. Warder, Chicago, IL (US); Lora A. Tucker, Lindenhurst, IL (US); Evelyn Mary McKeegan, Lake Forest, IL (US); Tamara J. Strelitzer, Racine, WI (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/486,233

(22) Filed: Jul. 13, 2006

Related U.S. Application Data
(63) Continuation-in-part of application No. 11/096,168, filed on Mar. 31, 2005, now abandoned.

(51) Int. Cl. C07K 1/12

(52) U.S. Cl. 530/414

(57) ABSTRACT

The subject invention relates to methods of profiling biomolecules from mammalian biological samples, for example, plasma or serum, in order to discover markers of disease, treatment, toxicity or efficacy. The resulting profiles may be used, for instance, to discover novel markers or patterns that may be used to classify samples into groups, for example, disease vs. normal, responders vs. non-responders, or to stratify patients for clinical trials.

### CROSS-REFERENCE TO RELATED APPLICATIONS

This application is a continuation-in-part of U.S. patent application Ser. No. 11/096168 filed Mar. 31, 2005, entitled “Methods for Biomarker Discovery and Diagnostic Screening”.

### TECHNICAL FIELD

The subject invention relates to methods for separating a particular biomolecule, e.g., a protein or biomarker, from other biomolecules in a biological specimen, especially where the other biomolecules are in relatively higher concentration than the particular biomolecule of interest. Methods according to the invention can be used to deplete a highly abundant protein comprising disulfide-linked cysteines in a biological fluid or cell culture media using a reducing agent. The present invention facilitates profiling biomolecules from mammalian or other biological samples, for example, from plasma or serum, in order to discover markers of disease, treatment, toxicity or efficacy.

### BACKGROUND OF THE INVENTION

Blood is the most widely sampled biological fluid for clinical diagnosis. In particular, the blood proteome (a collection of all of the proteins in blood) is an attractive resource useful for discovering biomarkers of disease and monitoring drug effects since this fluid is in contact either directly or indirectly with almost all of the cells in the human body. However, analysis of the plasma proteome is extremely challenging due to the high proportion of albumin (55%), the number and wide dynamic range of other proteins present and the heterogeneity of post-translationally-modified variants (Anderson et al., Mol Cell Proteomics 1(11):845-67 (2002)). In fact, the twelve most abundant proteins can account for >97% of the total plasma protein content.

Several strategies have been employed to remove albumin, IgG and other abundant proteins from serum and plasma. For example, Cibracon Blue dye chromatography is a common method to deplete albumin; however, this resin is known to interact with many other proteins that contain a nicotinamide adenine dinucleotide or purine dinucleotide-binding site (Gianazza et al., Biochem J 203(3):637-41 (1982); Thompson et al., Proc Natl Acad Sci USA 72(2):669-72 (1975)). In addition, bilirubin and fatty acids bound to albumin interfere with the albumin-Cibracon Blue interaction (Gianazza et al., supra (1982)). The variability of these lipids between patient blood samples potentially contributes to inconsistent removal of albumin.

The simultaneous depletion of several of the most abundant blood proteins, namely albumin, IgG, transferrin, haptoglobin, α-1-antitrypsin and IgA, can be achieved using affinity-purified polyclonal antibodies (pABs). Although this approach allows a more in-depth survey of the plasma proteome (Pieper et al., Proteomics 3(7):1345-64 (2003)), the reagents are not compatible across all species, the columns have a limited protein binding capacity and can only be used a finite number of times.

The discovery of plasma biomarkers relies on techniques that facilitate the detection and comparison of protein features. The most commonly employed profiling approaches for a native plasma sample include two-dimensional polyacrylamide gel electrophoresis (2D-PAGE) and Matrix-Assisted Laser Desorption Ionization Time-Of-Flight Mass Spectrometry (MALDI-TOF-MS). More recently, plasma samples have been subjected to protease digestion prior to Multidimensional Liquid Chromatography (MDLC)-MS/MS. This latter approach provides superior sensitivity but it is more difficult to monitor protein regulations and the natural proteolytic processing information is lost due to the digestion step. Regardless of the profiling method, only limited information about protein content in such samples is generated without depleting abundant protein(s) present.

Until recently, only the plasma proteome >10 kDa has been interrogated, due to the mass range limitations of 2D-PAGE. The development of mass spectrometry technologies, in particular MALDI-MS, has facilitated monitoring the plasma peptidome (<15 kDa). In general, MALDI-MS provides access to a large m/z range (500-200,000 Da), is easy to operate/automate and interpretation is relatively straightforward. However, disadvantages include the lack of direct protein identifications (except for small peptides), and the suppression of signal intensities when complex biological fluids, e.g., blood samples, are analyzed. Reduction or inhibition of protein ionizations is generally due to interfering species (salts, lipids, etc.) and abundant proteins (detector saturation and ion suppression). Surface Enhance Laser Desorption Ionization Time-Of-Flight Mass Spectrometry (SELDI-TOF-MS) partially circumvents the problems related to complex samples through the use of chromatographically-coated sample introduction devices (ProteinChips). This facilitates sample simplification and removal of contaminating salts. However, the surfaces have a limited binding capacity and the extra processing steps potentially contribute to assay variability.

There is growing evidence that abundant proteins and their exogenously-derived fragments have a potentially high predictive power in disease diagnosis. SELDI-TOF-MS combined with bioinformatic approaches, have led to the discovery of plasma profiles with putative masses below 15 kDa that serve to distinguish the onset of cancer (Petricoin et al., Lancet 359(9306):572-77 (2002); Adam et al., Cancer Res. 62(13):3609-14 (2002)). In addition, there is evidence that potentially useful clinical peptide markers may be bound to abundant proteins, such as albumin (Mehta et al., Dis Markers 19(1):1-10 (2003-2004); Zhou et al., Electrophoresis 25(9):1289-98 (2004)). Thus, more efficient strategies to investigate these bound peptides are warranted.

Sample preparation methods that do not require specialized reagents (antibodies, chemical resins, etc.) and facilitate depletion of abundant proteins would provide significant utility for analyte detection and proteomic profiling. In general, protein precipitation and ultrafiltration strategies that discriminate based on size/pI or size, respectively, have been applied as a step prior to profiling by gels or MS (Jiang et al., J Chromatogr A 1023(2):317-20 (2004); Gretzer et al., Prostate 60(4):325-31 (2004)). A precipitation strategy is probably preferable over ultrafiltration since the membranes used for ultrafiltration are not robust for the high protein concentration of plasma and even in a diluted state suffer from fouling (Velasco et al., J Colloid Interface Sci 266(1):148-52 (2003)).

The effect of temperature or chemical treatment (heat, metal, or organic) on aggregation or precipitation of protein standards and simple mixtures have been demonstrated. In particular, reducing agent mediated aggregation kinetics of bovine serum albumin have been investigated at protein concentrations significantly less, i.e., 9-12 fold less concentrated, than the amount commonly detected in plasma (45-60 mg/mL). In one example, as monitored by small angle x-ray diffraction (SAXS), a 5 mg/mL solution (i.e., an 8-fold dilution over the concentration of albumin in native plasma) of purified bovine serum albumin in the presence of excess (218-fold) dithiothreitol reducing agent began to form a soluble aggregate within 3 minutes and reached equilibrium in approximately 15 minutes (Ueki, et al., Biophys. Chem. 23(1-2):115-24 (1985)). The protein aggregates formed were reported to be soluble and predicted by the paper's authors to be only about 4-5 monomers per complex. No precipitation, concentration or separation of the soluble albumin aggregates from other solution components was reported.

Lodish, et al., J. Biol. Chem. 268(27):20598-20605 (Sep. 25, 1993) report addition of 2 mM dithiothreitol to human hepatoma cells blocking, in a reversible fashion, the secretion of albumin. Secretion is reportedly blocked by retaining cysteines in the protein in a reduced state so that normal disulfide bonds are not formed and the protein does not undergo intracellular folding and secretion from the cells. Instead, the protein exists in a soluble aggregated state within the cellular endoplasmic reticulum as long as dithiothreitol (DTT) is present and disulfide bonds cannot form.

Sawyer, et al., J. Biol. Chem. 269(35):22440-22445 (Sep. 2, 1994) report that treatment of HepG2 cells with a low concentration (e.g., 3 mM) of DTT results in the formation of soluble heterotypic complexes that can be enriched using antibody-based immunoprecipitation. The soluble aggregates reportedly form intracellularly. Sawyer, et al. found no evidence of aggregation of disulfide-linked proteins excreted from cells (or originating from any other biological samples) into media after reduction by DTT.

In view of the above, there is certainly an unmet need for methodologies to process plasma/serum or other blood component-containing formulations that facilitate the analysis of proteins and other biomolecules in blood, for analyte monitoring or profiling. In particular, no efficient way has existed to directly profile samples (without any preceding sample fractionation or enrichment) that are heavy laden with albumin (e.g., plasma or serum) by, for example, a mass spectrometry technique. In addition, it would be advantageous to have treatments of albumin-containing samples using a reducing agent under conditions that facilitate enrichment, and if desired, simple separation of this species into a particular phase.

All patents and publications referred to herein are hereby incorporated in their entireties by reference.

### SUMMARY OF THE INVENTION

The present invention encompasses methods for separating a biomolecule contained in a complex biological sample, e.g., to produce a phase enriched in the biomolecule, for biomarker discovery and diagnostic assays.

According to one embodiment, the present invention relates to a method for separating a first biomolecule in a biological specimen from a second biomolecule in the biological specimen, where the second biomolecule has a relatively lower concentration in the biological specimen than the first biomolecule. The biological specimen is reacted with a reducing agent, creating first and second phases, in which the first phase is relatively enriched in the first biomolecule and the second phase is relatively enriched in the second biomolecule. The first phase and the second phase are segregated to achieve separation of the first biomolecule from the second biomolecule. For example, one of the phases can be a liquid and the other phase can be a solid, which upon separation provide a supernatant and a pellet, respectively. The concentration of the first biomolecule in the biological sample should be sufficiently high to induce a precipitation. The content of each of these phases may be identified using conventional analytical techniques (e.g., MALDI-TOF-MS, SELDI, chromatography) which are well-known in the art.

In one aspect, the method provides a phase that is enriched in at least one biomolecule (e.g., a protein or peptide) of relatively lower abundance in the biological specimen. Once isolated using methods according to the present invention, the biomolecules of relatively lower abundance can be analyzed by any of a wide variety of conventional analytical methods, especially those useful to proteomics, including MALDI-TOF MS, SELDI, chromatography, etc.

In another aspect, the method of the present invention provides a precipitate containing a biomolecule of relatively lower abundance that is associated with a more abundant biomolecule. The association can be physical (e.g., adhesion, adsorption, entrapment of a peptide within the three-dimensional structure of an abundant protein, or other affinity or binding type) or chemical (e.g., ionic bond).

Preferably, the creation of the precipitate takes place within an hour after mixing the reducing agent and the biological specimen. More preferably, the precipitate is created within 30-45 minutes after mixing the reducing agent and the biological specimen. This advantageous and practical timeline can be accomplished when the concentration of the relatively more abundant biomolecule is sufficiently high in the biological specimen. That is, the speed of the precipitation reaction is concentration-dependent. A sufficiently high concentration of the relatively more abundant biomolecule can be achieved by a) avoiding adding too large of a volume of reducing agent to the biological specimen; b) using a reducing agent at a relatively high concentration; c) and concentrating relatively dilute samples using standard laboratory techniques such as ultrafiltration or lyophilization.

In another aspect of the invention, the biological specimen can be naturally occurring (e.g., native, that is, as derived directly from a living organism) or made or formulated by humans. Biological specimens suitable for use in the present invention are preferably directly or indirectly derived from a mammal. More particularly, biological specimens useful in the present invention include serum, whole blood, plasma, urine, cerebral spinal fluid, tears, semen, aqueous humor and intestinal fluids. In some circumstances, it may be desirable to concentrate the contents of a given biological specimen to provide suitably high concentrations of biomolecules of interest for use in methods according to the present invention. In addition, blood-component (e.g., serum)-containing formulations such as cell culture supernatant and samples stabilized with serum can be used.

In another aspect of the invention, the biomolecule in relatively high concentration in the biological specimen is a protein having disulfide-linked cysteines. In blood-related or blood-derived specimens, for example, such proteins include albumins, globulins, fibrinogens, hemoglobins, and other types.

Suitable reducing agents useful in the methods of the present invention include those selected from the group consisting of tris(2-carboxyethyl)phosphine (TCEP), dithiothreitol (DTT) and β-mercaptoethanol. (TCEP is available as a hydrochloride salt or in a pH neutralized form. Either of these forms is suitable.) These agents break disulfide bonds linking cysteines, which, upon reduction, expose sites, e.g., hydrophobic regions, on the protein molecule that are prone to aggregate.

The subject invention also encompasses a method of reducing or depleting the amount of at least one highly abundant protein comprising at least one disulfide-linked cysteine bond in a biological sample. This method comprises the steps of mixing the sample with a reducing agent in an amount sufficient to form a protein precipitate of the at least one highly abundant protein and removing that protein precipitate from the sample.; depositing the mixed (i.e., reduced) sample on a target; drying the mixed sample; applying a matrix to the mixed sample; drying the matrix; applying a source of photo-excitation to the dried matrix; and profiling the sample in order to ensure reduction in the amount of albumin contained in the sample.

The biological specimen may be, for example, serum, whole blood, plasma, urine, cerebral spinal fluid (CSF), tears, semen, aqueous humor, intestinal fluid (e.g., bile), cell culture medium, tissue culture medium and is preferably obtained from a mammal. The fluid comprises at least one biomolecule such as, for example, a peptide, a protein, a nucleic acid, a small organic or inorganic molecule that may ionize in a mass spectrometer. Also, the reducing agent may be, for example, tris(2-carboxyethyl)phosphine (TCEP), dithiothreitol (DTT) or β-mercaptoethanol, as described above. (However, other reducing agents may also be utilized.)

The reducing agent, when mixed with the biological sample, is in a final concentration of at least about 5 mM and at most about 50 mM. Integers within this range are considered to fall within the scope of the invention.

As set forth above, methods according to the present invention can be used to prepare a specimen analyzed with a mass spectrometry technique. In accordance with these embodiments, the target is a Matrix-Assisted Laser Desorption Ionization Time-Of-Flight Mass Spectrometry (MALDI-TOF-MS) sample introduction device or a Surface-Enhanced Laser Desorption Ionization Time-Of-Flight Mass Spectrometry (SELDI-TOF-MS) sample introduction device.

The specimen to be analyzed can be dried, such as where drying is accomplished through application of a source selected from the group consisting of vacuum, air and heat. Thus, the profiling step can be accomplished using a Matrix-Assisted Laser Desorption Ionization Time-Of-Flight Mass Spectrometry (MALDI-TOF-MS) instrument. The matrix may be, for example, alpha-cyano-4-hydroxycinnamic acid (CHCA), sinapinic acid (SPA) or 2,5-dihydroxy benzoic acid (DHB). Further, photoexcitation of the matrix results in protonation or deprotonation of biomolecules in the biological fluid sample.

Additionally, the present invention includes another method of depleting the amount of albumin contained in a sample of a biological fluid. This method comprises the steps of mixing the sample with a reducing agent; incubating (e.g., in a test tube) the mixed sample until formation of a precipitate occurs; centrifuging or, via another means, separating the incubated sample; recovering the supernatant from the resulting centrifuged sample; depositing said centrifuged sample on a target; drying the centrifuged sample; applying a matrix to the centrifuged sample; drying the matrix; applying a source of photo-excitation to the dried matrix; and profiling the centrifuged sample in order to ensure reduction in the amount of albumin contained in the sample. The biological fluid, content and source thereof, reducing agent, concentration of reducing agent, target, drying step, matrix, profiling and photoexcitation are as defined in the method above.

The present invention includes methods for discovering a biomarker (e.g., concentration or modification of an endogenous biomolecule or possibly identification of a substance that is unique to a particular disease) associated with a biological state of interest, such as the presence or absence of a disease condition or stage of disease, toxicity or efficacy of drug treatment. Exemplary modifications of biomolecules which can be observed include phosphorylation, glycosylation and proteolytic processing. In these methods, a plurality of biological specimens is provided, where each specimen represents either the presence or absence of the biological state of interest. Each specimen is treated with a reducing agent to form a precipitate from the treated specimen. The precipitate (e.g., pellet) or supernatant or both can be analyzed to provide a profile of biomarker content in each treated specimen. Biomarkers can be identified by examining those biomolecules that are differentially regulated as a result of the biological states of interest represented by the biological specimens treated.

### BRIEF DESCRIPTION OF THE DRAWINGS

FIG. 1 illustrates the effect of the reducing agent TCEP on the MALDI-TOF-MS spectra of human serum. (a) The low m/z range of the MALDI-TOF-MS spectra revealing that TCEP treatment results in the peptide-rich detection. (b) The 20-80 kDa range of the same MALDI-TOF-MS spectra that clearly shows the suppression of the albumin signal upon TCEP treatment concomitant with the increase in protein signals.

FIG. 2 illustrates the reproducibility of MALDI-TOF-MS peak intensities for mouse plasma treated with TCEP. (A) MALDI-TOF-MS profiles (3-7 kDa region) of the same mouse plasma sample (4 replicates). (B) Statistical evaluation of the peaks annotated in FIG. 2A.

FIG. 3 illustrates the effect of TCEP (25 mM) on the distribution of a fluorescent peptide in plasma. Plasma was spiked with a FITC-labeled peptide (720 ng/μL). A) untreated plasma; B) supernatant of TCEP-precipitated plasma. In the absence of TCEP, peptide is concentrated at the outer edge of the sample well and not accessible for analysis. TCEP treatment results in a homogeneous distribution of the fluorescent peptide.

FIG. 4 illustrates anti-albumin Western Blot analysis of TCEP-treated plasma. Lane 1. Native plasma standard (0.1 μL plasma equivalent); Lane 2. Supernatant of TCEP-treated plasma (0.1 μL plasma equivalent); Lane 3. Native plasma standard (0.004 μL plasma equivalent); Lane 4. Supernatant of TCEP-treated plasma (0.004 μL plasma equivalent).

FIG. 5 illustrates the effect of reducing agent on the MALDI-TOF-MS spectra of plasma spiked with the recombinant protein Kringle 5 (rK5). (A) In the absence of reducing agent, the predominant peak originates from albumin (black trace). In the presence of DTT, the albumin peak is suppressed and a significant number of features are detectable. (B) Expanded region of FIG. 1A reveals that reducing agent is essential for the detection of rK5 in plasma.

FIGS. 6a-6b are one-dimensional SDS-PAGE analyses of human plasma samples depleted with the reducing agent mediated approach of the invention using TCEP compared with treatments using commercially available kits.

FIGS. 6c and 6d are Western blots to quantify the relative amounts of analyte (transferrin or plasminogen, respectively) in human plasma sample, pellet and supernatant.

FIG. 7 is a Venn diagram comparing the number of protein identifications from MuDPIT analyses of organic precipitation, immunoaffinity depletion and reducing agent mediated precipitation (RAMP) methods of sub-proteome generation.

FIG. 8 is a one-dimensional SDS-PAGE analysis identifying proteins enriched in a solid pellet provided from treating a human plasma sample using reducing agent mediated precipitation (RAMP) in a method according to the present invention.

FIG. 9 illustrates the effect of DTT on the MALDI-TOF-MS spectrum of an elution from an affinity purification of GAPDH that is contaminated with albumin.

FIG. 10 illustrates the effect of TCEP on the MALDI-TOF-MS spectrum of patient serum. A 7.7 kDa diagnostic biomarker is detectable only in the presence of reducing agent.

FIG. 11 illustrates representative plasma profiles from a MetAP2 inhibitor study. In particular, a plasma sample (4.5 μL) from a tumor-bearing mouse (vehicle treated) was diluted with reducing agent (0.5 μL of 250 mM TCEP). The sample was incubated at ambient temperature for 45 min., centrifuged for 10 min at 20,000×g. The supernatant (1.0 μL) was spotted on a SELDI-MS ProteinChip™ (H50, reverse phase) and air-dried. Sinapinic acid (1.0 μl) was applied, and after air-drying, was analyzed in a Ciphergen™ PBSIIc MALDI-TOF-MS.

FIG. 12 illustrates discriminant analysis of plasma profiles from tumor-bearing (B16F10) mice treated with vehicle or the MetAP2 inhibitor TNP-470. Canonical plot for all replicates (5) for each mouse plasma sample. Large icons denote a misclassification.

FIG. 13 illustrates regulations for each of the discriminating markers from the MetAP2 inhibitor study. The numeric labels on the X-axis refer to the mass/charge (m/z) of the peptides or proteins. (The underscore represents a decimal point.)

### DETAILED DESCRIPTION OF THE INVENTION

The present invention encompasses methods for separating a biomolecule contained in a complex biological sample, e.g., to produce a phase enriched in the biomolecule, for biomarker discovery and diagnostic assays.

The subject invention also relates to methods that deplete proteins, and in particular embodiments, relatively highly abundant proteins comprising at least one disulfide-linked cysteine bond, via reducing agent treatment. Specifically, this invention significantly increases the types of biomolecules (for example, peptides, proteins, nucleic acids, small molecules, etc.) that can be detected by MALDI-TOF-MS (or other analytical technique, in a sample containing, for example, the protein albumin or other highly abundant proteins.

A particularly advantageous utility is recognized for treating samples such as plasma or serum. For example, a highly abundant protein such as albumin (and its isoforms) constitutes greater than 40% of the protein content of plasma or serum. Attempts to analyze serum samples directly by MALDI-MS have been hampered by the fact that very few peaks are detected and the majority of these peaks originate from intact albumin monomers. However, treatment of the sample with reducing agent, according to the present invention, as illustrated below, before mass spectrometry (for example) analysis results in the suppression of the albumin (and/or other proteins, including highly abundant proteins comprising at least one disulfide-linked cysteine bond) signal by MALDI-MS and allows less abundant peptides and proteins to be monitored.

Preferably, the reducing agent concentration is sufficient to form a protein precipitate when reacted with the biomolecule of relatively high abundance in the biological sample. Common laboratory reducing agents include, but are not limited to: tris(2-carboxyethyl)phosphine (TCEP), for example using its hydrochloride salt or a pH neutral form, dithiothreitol (DTT), and β-mercaptoethanol.

The amount of reducing agent needed to achieve precipitation depends on the amount of highly abundant protein(s) present in the sample to be treated, but can also be determined readily by those of ordinary skill in the art using conventional techniques (e.g., by titration). Generally, the higher the concentration of the “abundant” protein, the more reducing agent will be needed to achieve the desired effect. The stock solution of reducing agent may be a dilute or concentrated solution. In addition, the reducing agent may be added directly to the biological sample or alternatively, the sample may be added to the reducing agent (i.e., the order of contacting the reducing agent and the sample is not important.) By way of example, a sample may be diluted minimally from its natural state (e.g., 9 parts albumin-containing sample plus 1 part reducing agent) with a concentrated solution of reducing agent in a range of approximately 50-500 mM. Preferably, since the rate of precipitation depends on the concentrations of the biomolecule (e.g., albumin) in the sample to be treated and of the reducing agent, the sample is minimally diluted before treatment with the reducing agent and a final concentration of reducing agent in the sample/reducing agent mixture is in the range of 5-50 mM, although in a particular circumstance, either the concentration of the biomolecule or the reducing agent concentration may be enough to induce precipitation

The methods of the present invention generally involve contacting a complex biological specimen such as, for example, serum, plasma, urine, cerebral spinal fluid (CSF), tissue culture media, cell culture media, or any solution containing albumin or another biomolecule in a sufficient amount to precipitate-with a reducing agent.

The mixture is then briefly mixed (2-5 s) by vortexing or manually agitating the vial.

Once the precipitate has formed, the sample is further treated so as to separate the precipitate. This separation step may be achieved by any means well known to those skilled in the art for removing protein precipitates. For example, the sample comprising the protein precipitate may be centrifuged.

In another embodiment, the reducing agent/sample mixture is spotted on a MALDI or SELDI sample introduction device often referred to as a “target”. Typically, a target is a flat metal surface (e.g., gold, steel, aluminum, etc.) with wells for sample deposition. The sample is allowed to dry via air or via application of a heat source or vacuum. Normally, 1 μL per analysis spot is utilized; however, one typically uses between 0.5-5 μL. Sample aliquots greater than 5 μL form thick cake structures that have a tendency to detach from the surface.

As a next step, an energy-absorbing molecule, commonly referred to as “matrix” is added (typically 0.5-1.0 μL) to the spot and allowed to dry via air or via application of a heat source or vacuum. A MALDI matrix is a small organic molecule that exhibits a strong absorbance at the selected laser wavelength of the instrument and is low enough in mass to be sublimable. The analyte molecules (e.g., protein, nucleic acid, small molecules, etc.) become ionized by protonation (positive-ion mode) or deprotonation (negative-ion mode) from the photo-excited matrix. The mechanisms of the MALDI process are not totally elucidated, and the ionization process may occur through different mechanisms. Typical matrices include, but are not limited to, alpha-cyano-4-hydroxycinnamic acid (CHCA), sinapinic acid (SPA) and 2,5-dihydroxy benzoic acid (DHB).

Alternatively, the second step (referred to above as direct deposition of the sample and reducing agent mixture) involves the incubation of the mixture until a significant precipitation is visible. This step is generally carried out at ambient temperature to accomplish the desired rate of precipitation. Performing the reaction at higher temperature or reducing agent concentrations (e.g., >1 M TCEP) generally drives the reaction very quickly and leads to an extremely large pellet with minimal recovery of supernatant. (It is noted, however, that too much reducing agent can create a solid phase (e.g., pellet) of such size that a relatively small amount of supernatant is formed, which may adversely affect analysis of the latter phase.) Typically, the precipitation reaction is allowed to proceed for 30-60 min. The sample is centrifuged at 16-20,000×g for 10 min., and the supernatant (0.1-5 μL) spotted on a MALDI-MS or SELDI-MS target. Matrix is added prior to collection of the MALDI-MS spectra.

Next, the sample is analyzed in a MALDI-TOF-MS. More specifically, spectral data can be collected on a variety of MALDI-TOF-MS instruments. These include, but are not limited to, PBS-II/PBS-IIc (Ciphergen Biosystems), Voyager-DE (Applied Biosystems) and Ultraflex (Bruker).

As indicated in the examples below, typically, data were collected on a Ciphergen Biosystems PBS-IIc (Fremont, Calif.) mass spectrometer due to the proven reproducibility and ease of implementation. For example, mass spectra were recorded in linear positive ion mode at source voltage of 20 kV and a 1 GHz digitizer rate. Each spot was sampled using the same protocol: position 30 through 70 (0-100 scale), with 2 warming laser shots (not included in the average) and 8 laser shots (included in the average). The laser intensity was fixed at 220 (0-300 scale) and the detector sensitivity at 6 (0-10 scale). (Note: The laser intensity and detector sensitivity are empirically determined and generally vary between instruments.) The PBSIIc MS was externally calibrated using the “All-In-One” peptide mass standard from Ciphergen Biosystems, Inc. (Fremont, Calif.).

The spectra may be processed using the Ciphergen ProteinChip software. Typically this includes implementing a baseline correction algorithm and peak detection.

With respect to the benefits of the non-incubation method of the invention, it significantly decreases the ionization signal of albumin observed when the sample is analyzed by MALDI-TOF-MS. It also enhances the signal-to-noise ratio of the MALDI-TOF-MS spectra and increases the number of observed peptide and protein features. Further, it improves reproducibility and reduces localization of protein clusters (i.e., hot-spots of ionization) compared to a conventional MALDI-TOF-MS analysis of a complex sample, and it is also species independent. (For example, the albumin signal is suppressed for the following plasma samples: human, monkey, bovine, canine, rat and mouse.)

In terms of the preferred incubation method of the present invention, it results in the formation of an insoluble pellet that is significantly enriched with albumin. Further, it facilitates >99% depletion of albumin from the supernatant of human plasma and serum as determined by an ELISA. However, albumin fragments are still detectable by MALDI-TOF-MS. Further, the method is reproducible, as determined by MALDI-TOS-MS spectra, HPLC profiles of the abundant UV absorbing species, extent of albumin depletion and the volume of supernatant recovered from replicate experiments. Moreover, the method also removes approximately 20-30% additional plasma/serum proteins from the sample, either through a precipitation mechanism or trapping in the pellet. Finally, unlike other methods of albumin depletions (e.g., affinity-dye chromatography or immunodepletion), this method does not result in sample dilution or suffer from buffer incompatibility, is readily scalable to >ml quantities and is not dependent on specialized reagents.

Additional benefits of the methods of the present invention include the fact that processing is simple and amenable to automation. In the case where the mixture of the sample and the reducing agent is immediately spotted on the MALDI-MS target, albumin is not depleted from the sample, therefore potentially reducing loss of peptides and proteins that are bound to albumin. These proteins would have otherwise been degraded or filtered from the blood, therefore providing a possible enrichment mechanism for lower abundance species (Mehta et al., Dis Markers 19(1):1-10 (2003-2004; Zhou et al., Electrophoresis 25(9):1289-98 (2004)).

Additionally, the versatility and reproducibility of this method allows for its application in a variety of research areas such as, for example, sample preparation for small molecule analysis by MS or as a first step in purification or profiling of biomolecules.

With respect to the uses of the present invention, it should be noted that the above-described methods might be applied widely, including the discovery and identification of biomarkers that aid in the transition and development of drug candidates. The markers include, but are not limited to: 1) pharmacodynamic biomarkers of target stimulation or inhibition in order to establish appropriate dosing regimens in humans and to demonstrate separation between maximum effective and maximum tolerable dosing in Phase I studies; 2) early efficacy biomarkers for patient monitoring, in order to make better clinical decisions; 3) stratifying biomarkers allowing predictive classification of patient response to therapy; and 4) developing diagnostic profiles for disease staging and early detection. Ideally, profiles generated from the MALDI-TOF-MS spectra may be used directly as multiple marker panels to correctly classify animals or patients (e.g., disease, response to therapy, etc.). However, it is more likely that identification of the discriminating features will facilitate the application of other high throughput formats, such as enzyme immunosorbent assays (ELISA) or liquid chromatography mass spectrometry (LC-MS), that are validated clinical technologies.

For example, the present invention includes methods for discovering a biomarker (e.g., concentration or modification of an endogenous biomolecule or possibly identification of a substance that is unique to a particular disease) associated with a biological state of interest, such as the presence or absence of a disease condition or stage of disease, toxicity or efficacy of drug treatment. Exemplary modifications of biomolecules which can be observed include phosphorylation, glycosylation and proteolytic processing. In these methods, a plurality of biological specimens is provided, where each specimen represents either the presence or absence of the biological state of interest. Each specimen is treated with a reducing agent to form a precipitate from the treated specimen. The precipitate (e.g., pellet) or supernatant or both can be analyzed to provide a profile of biomarker content in each treated specimen. Biomarkers can be identified by examining those biomolecules that are differentially regulated as a result of the biological states of interest represented by the biological specimens treated.

The present invention may be illustrated by the use of the following non-limiting examples:

### EXAMPLE I

### Reducing Agent Treatment of Human and Rodent Plasma Samples Increases the Data Content and Reproducibility of MALDI-MS Profiles

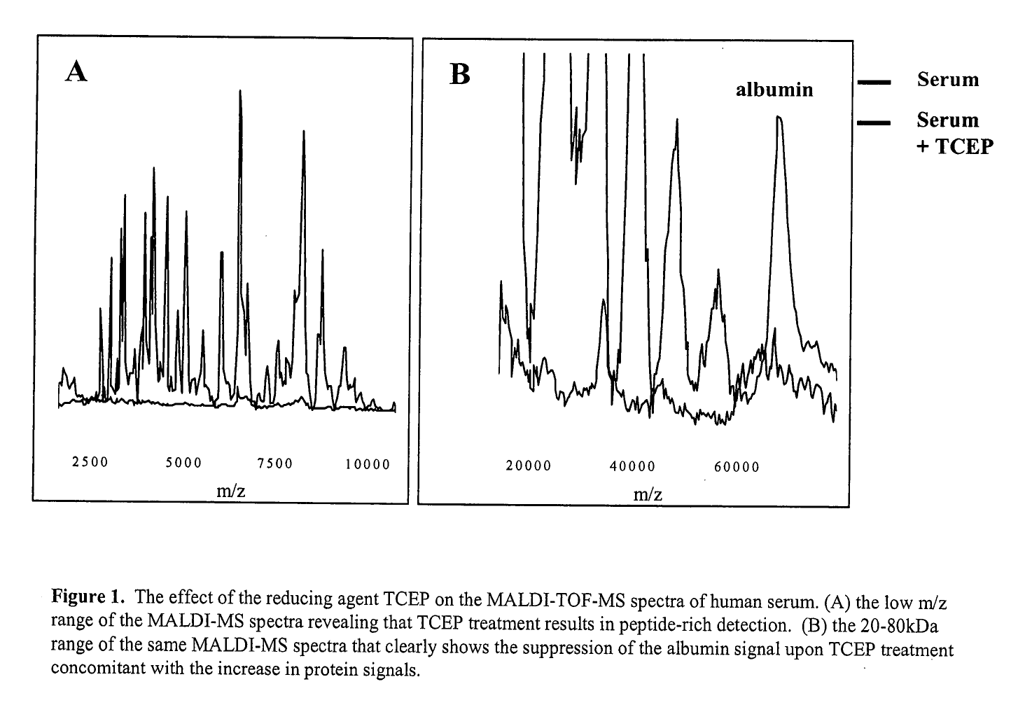

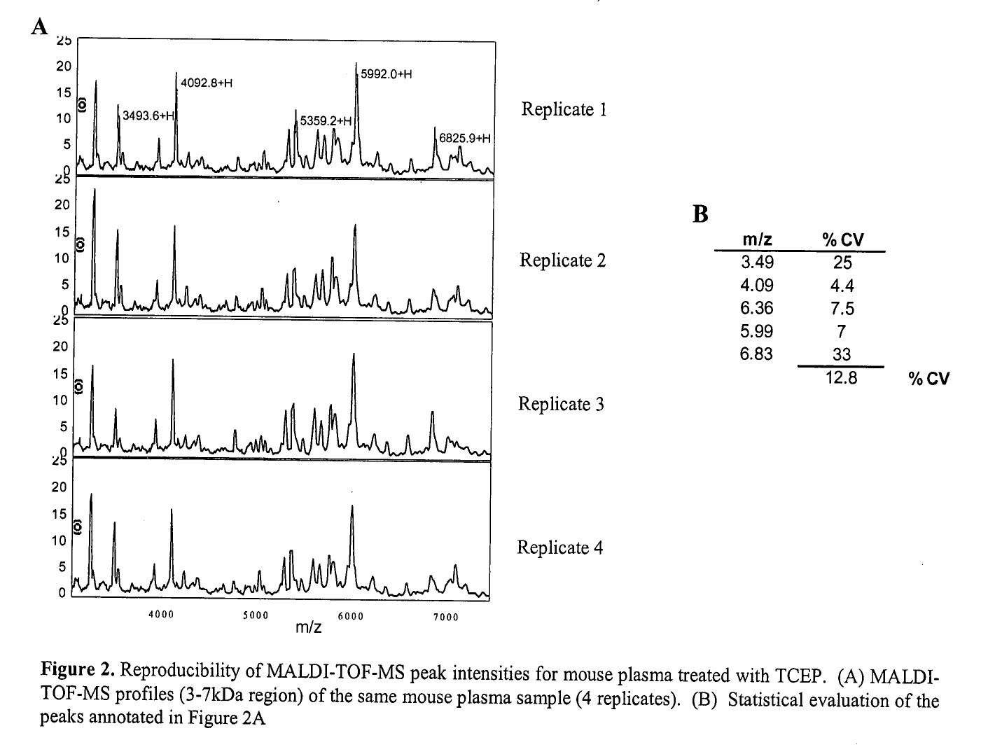

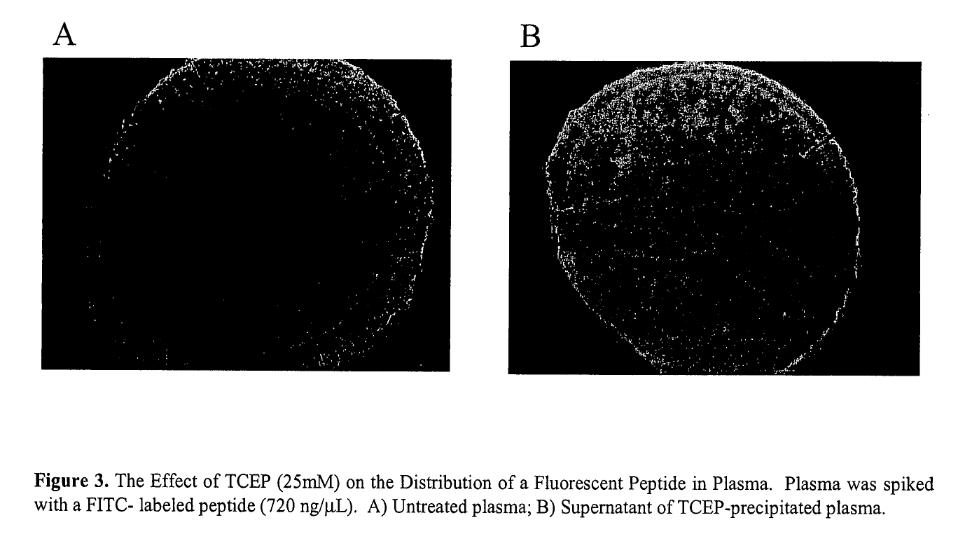

The treatment of plasma or serum from all species tested, in particular human and mice, revealed a significant increase in the number of peaks observed in MALDI-MS spectra. An example of the low m/z region of a MALDI profile of human serum sample is presented in FIG. 1a. This observation of enhanced signal at low m/z (small peptide region) is accompanied by a reduction in the ionization of albumin monomers at approximately 66,000 m/z (FIG. 1b). The highly reproducible nature of this process using a mouse plasma sample is presented in FIG. 2a/FIG. 2b. Since the crystallization of the MALDI-MS matrix is a major determinant of the reproducibility, the effect of the reducing agent on the crystallization process and the distribution of a control peptide was assessed using a fluorescent probe (FIG. 3). It was observed that the presence of reducing agent provided a more homogeneous distribution of the peptide presentation.

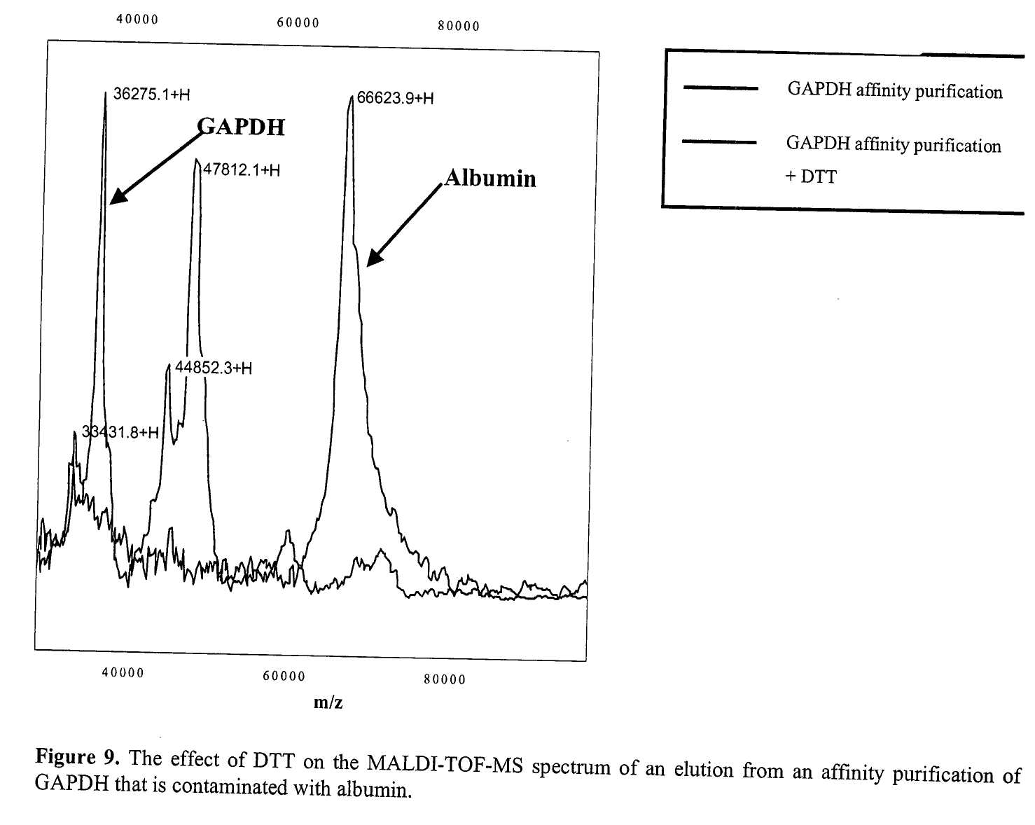

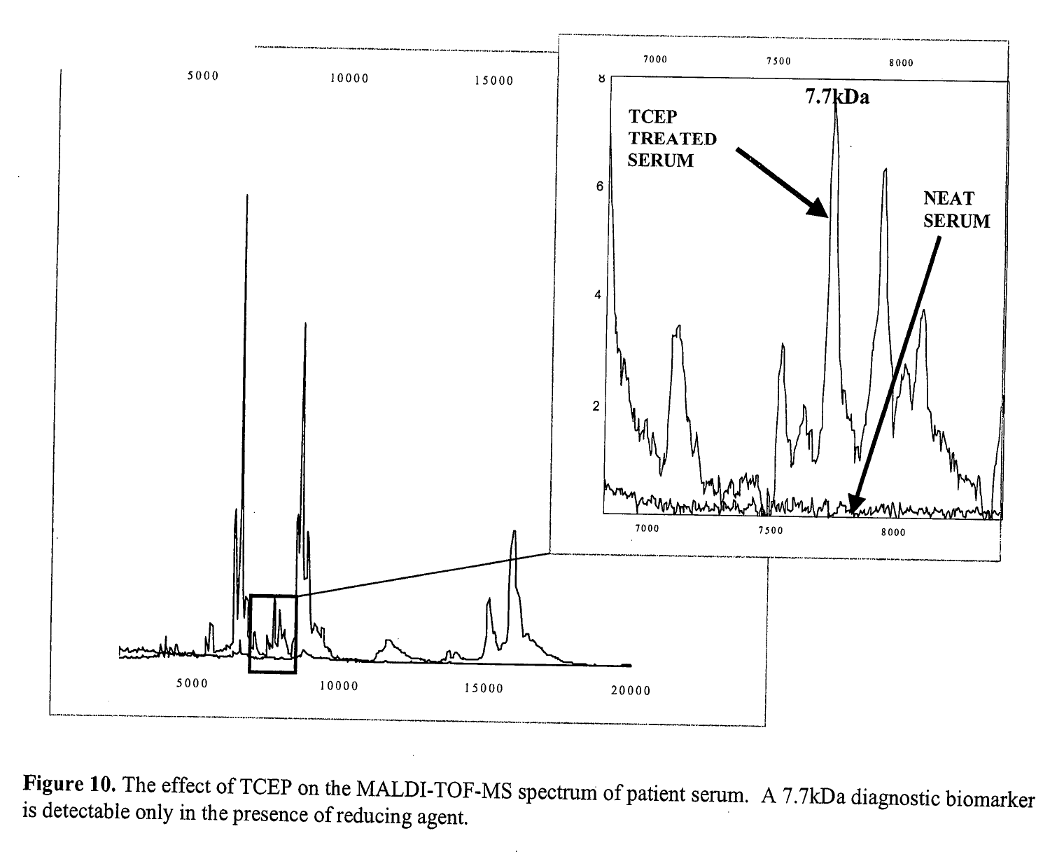

An example of the effect of reducing agent treatment on a non-plasma or serum sample is presented in FIG. 9. In this case, the detection of an analyte was suppressed by the presence of albumin (and potentially other unknown serum components). Post-treatment with reducing agent, the analyte, GAPDH, was clearly detected. Finally, the detection of a human serum protein that distinguishes one biological state of patients from another (putative biomarker) was accomplished. The 7.7 kDa peak is shown in FIG. 10.

### EXAMPLE II

### Analysis of r-K5 Present in Human Plasma

The following chemicals and reagents were utilized in the method:

Tris(2-carboxyethyl)phosphine hydrochloride (TCEP), Sinapinic Acid, Dithiothreitol (DTT), and Trifluoroacetic acid were obtained from Sigma-Aldrich (St. Louis, Mo.). Acetonitrile was obtained from EMD Chemicals Inc (Gibbstown, N.J.). The stock solutions of rK5 and ¹⁵N-rK5 were obtained from Abbott Laboratories (Abbott Park, Ill., USA). All water was distilled and deionized (resistivity >18.0 Megohm.cm).

Frozen aliquots of human citrated plasma were thawed for preparing the rK5 and ¹⁵N-rK5 standards. A 50 ng/μL r-K5 standard was 2-fold serially diluted with plasma spiked with 25 ng/μL ¹⁵N-rK5. This provided a series of standards from 50 ng/μL to 340 pg/μL rK5, all with a 12.5 ng/μL final concentration of ¹⁵N-rK5.

Typical experiments were conducted using freshly prepared DTT (250-500 mM) and/or TCEP (250 mM) in ddH₂O. Plasma (9 μL) was mixed with reducing agent (1 μL) and vortexed briefly. For the non-incubation experiment, 0.5-1.0 μL was spotted on a gold target or H50 ProteinChip array (Ciphergen Biosystems Inc., Fremont Calif.). Samples were air-dried and 1.0 μL of matrix (Sinapinic Acid in 50% AcN/ddH₂O, 0.1% TFA) was added to each sample. For the incubation approach, the plasma/reducing agent mixture was allowed to form a viscous white precipitate (generally 30-60 min). The sample was centrifuged (16-20,000×g, 10 min) prior to spotting the supernatant (0.5-1.0 μL) on a MALDI target.

Data were collected on a Ciphergen Biosystems PBS-IIc (Fremont, Calif.) SELDI-TOF-MS. Mass spectra were recorded in linear positive ion mode at source voltage of 20 kV and a 1 GHz digitizer rate. Each spot was sampled using the same protocol: position 30 through 70 (0-100 scale), with 2 warming laser shots (not included in the average) and 8 laser shots (included in the average). The laser intensity was fixed at 220 (0-300 scale) and the detector sensitivity at 6 (0-10 scale). The PBSIIc TOF MS was externally calibrated using the “All-In-One” peptide mass standard from Ciphergen Biosystems, Inc. (Fremont, Calif.). In the case where the ¹⁵N-rK5 IS was present, all spectra for a particular experiment were normalized to this peak intensity. Samples without an IS were normalized to total ion current starting at an m/z of 2000. Peak intensities for r-K5 in each spectrum were exported to MICROSOFT Excel™ for plotting and analysis.

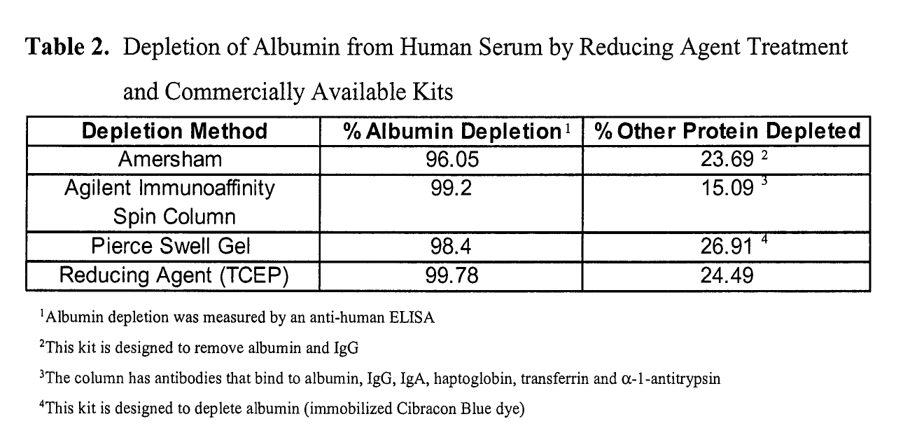

In view of the results obtained, a DTT/plasma solution was spotted on a MALDI-MS target at various DTT concentrations, and it was determined that >5 mM of reducing agent was sufficient for suppression of the albumin signal (see FIG. 5). Initially, the mechanism of ion suppression was unclear until it was observed that plasma/DTT samples formed a solid white precipitate in a microcentrifuge tube after 30-60 min at room temperature. The absence of an albumin signal in the supernatant was confirmed by spotting and analyzing on a MALDI-TOF-MS target.

The experiment was repeated with TCEP, another common laboratory reducing agent that is more reactive than DTT and is insensitive to pH. The detection of an rK5/¹⁵N-rK5 mixture spiked into plasma was dependent on reducing agent, likely due to the suppression of the albumin signal. In addition, TCEP-treated samples produced MALDI-TOF-MS spectra of higher resolution in the peptide region than their DTT counterparts, as demonstrated for the rK5 signals. The mechanism mediating the suppression of albumin ionization appears to be species-independent since plasma and serum from mouse, rat, dog, bovine and monkey, responded similarly to the human samples (data not shown).

### EXAMPLE III

### Identification and Monitoring of Protein Analytes in Plasma After Reducing Agent Treatment

Direct profiling or monitoring of protein analytes in plasma can be accomplished advantageously using methods according to the present invention. Frozen aliquots of human citrated plasma samples, reagents and instrumentation as described in Example I were used.

Using an AGILENT Multiple Affinity Removal System (MARS) spin column (commercially available from Agilent, Santa Clara, Calif.), 10 μL human plasma was diluted in 200 μL of buffer supplied with the kit and filtered through a 0.22 μM spin filter. The diluted and filtered plasma sample was applied to the spin cartridge and the flow-through collected. 10 μg of the initial filtrate (F1) was used directly for SDS-PAGE.

Using the Albumin and IgG Removal Kit (commercially available from GE Healthcare), 15 μL of human plasma was mixed with a suspension of affinity resin (750 μL) for 30 min at room temperature. The resin-plasma mixture was transferred to a spin column and the filtrate collected (˜500 μL). The sample was diluted to 10 μg and used directly for SDS-PAGE.

Using a PIERCE SwellGel Blue Albumin Removal kit (commercially available from Pierce, Rockford, Ill.), 25 μL of plasma was diluted with 25 μL of buffer supplied with the kit. A SwellGel disc was hydrated with 380 μL of ddH₂O and the excess liquid removed by centrifugation. The dilute plasma sample was spun through the spin column and then re-applied to the resin for a second binding event. Wash buffer supplied with the kit was applied to the column (2×125 μL) and the filtrate collected. The sample was diluted to 10 μg and used directly for SDS-PAGE. Crude plasma, protein depleted plasma, and the RAMP pellets were run on a 4-12% Nupage Bis-Tris gel (commercially available from Invitrogen) for 35 min at 200 V using a MES buffer (Invitrogen). All gels were fixed with 50% methanol/7% acetic acid for 30 minutes and then stained with GelCode Blue (Pierce). Images were captured using a FUJIFILM FLA-5000 imager.

Digestion of gel-slices and plasma pellets. A slightly modified procedure that was originally developed by Shevchenko et al. (Shevchenko, 1996) was employed for in-gel digestion. For in-solution digestion precipitated plasma pellets were solubilized in Urea, reduced and alkylated, and subsequently digested (Washburn 2001).

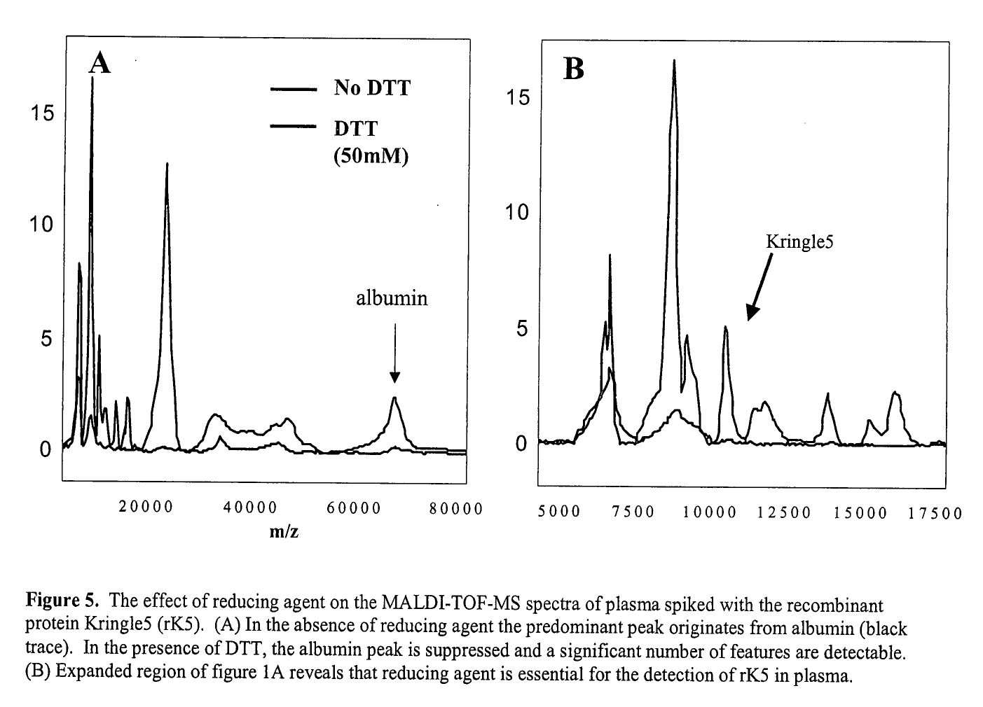

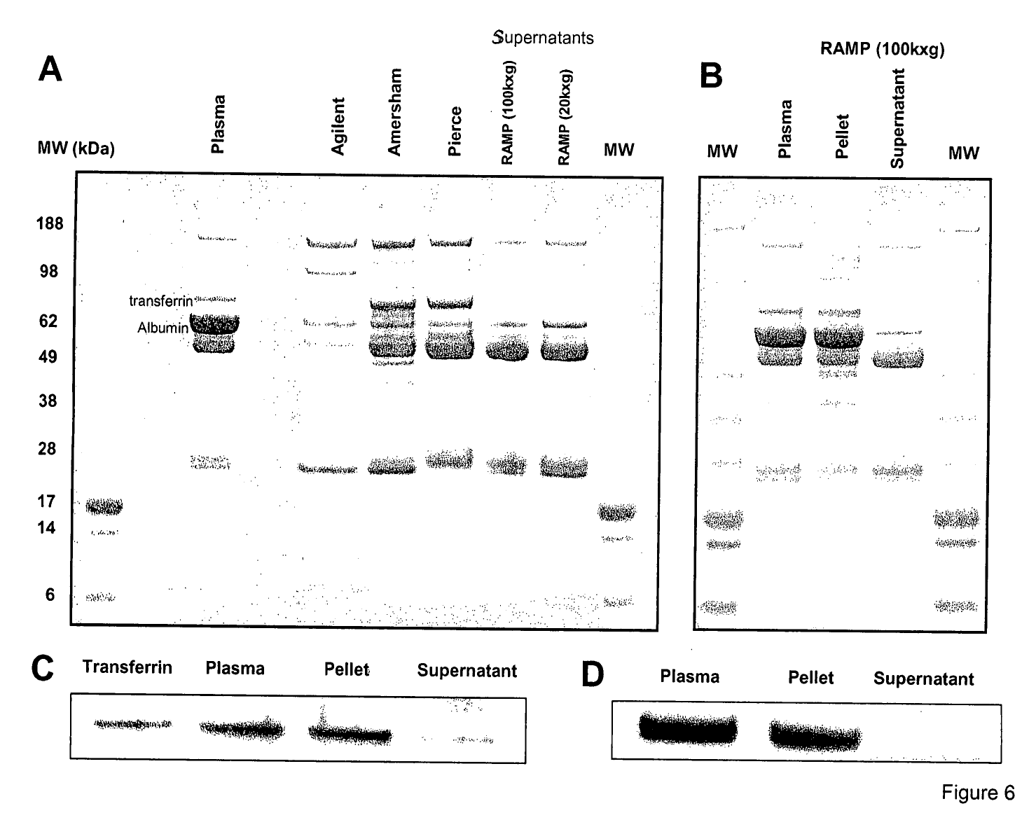

The partitioning of proteins between the pellet and supernatant was assessed using 1D-SDS-PAGE and Western blotting. A significant reduction in the albumin band intensity was observed for the supernatant from the TCEP-treated plasma (FIG. 6a). Reducing agent mediated albumin depletion was comparable to commercially available albumin depletion kits. These kits included a Cibacron blue dye swell-gel resin kit (Pierce, Rockford, Ill.) that removes only albumin, an albumin/IgG removal kit (GE Healthcare) and antibody cross-linked beads from a Multiple Affinity Removal System (MARS) kit (Agilent, Santa Clara, Calif.) used to deplete albumin, IgG, IgA, antitrypsin, haptoglobin and transferrin.

In addition, the supernatant from the TCEP-treated plasma exhibited a strong reduction in the intensity of a band at approximately 80 kDa that is present in the native plasma and pellet samples (FIG. 6b). This protein was identified by mass spectrometry as transferrin and confirmed using Western blotting (FIG. 6c). A similar trend was also observed for plasminogen (FIG. 6d). Routine recoveries of supernatant proteins are 25-30 mg/mL from plasma samples (˜75 mg/mL total protein).

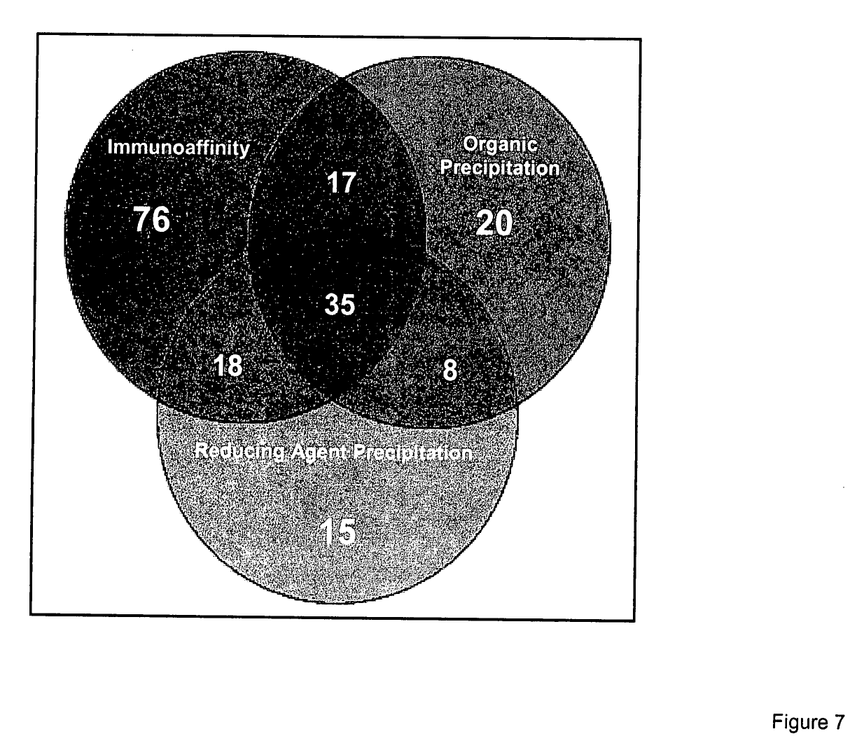

Identification of proteins in the RAMP supernatant revealed that this method is complementary to an organic precipitation and an immunoaffinity depletion column. These data are summarized in the Venn diagram of FIG. 7, where the numbers appearing in the figure represent the number of protein identifications made by the indicated method. The larger the number of protein identifications observed with the antibody depletion column is due to the depletion of a greater proportion of the abundant proteins.

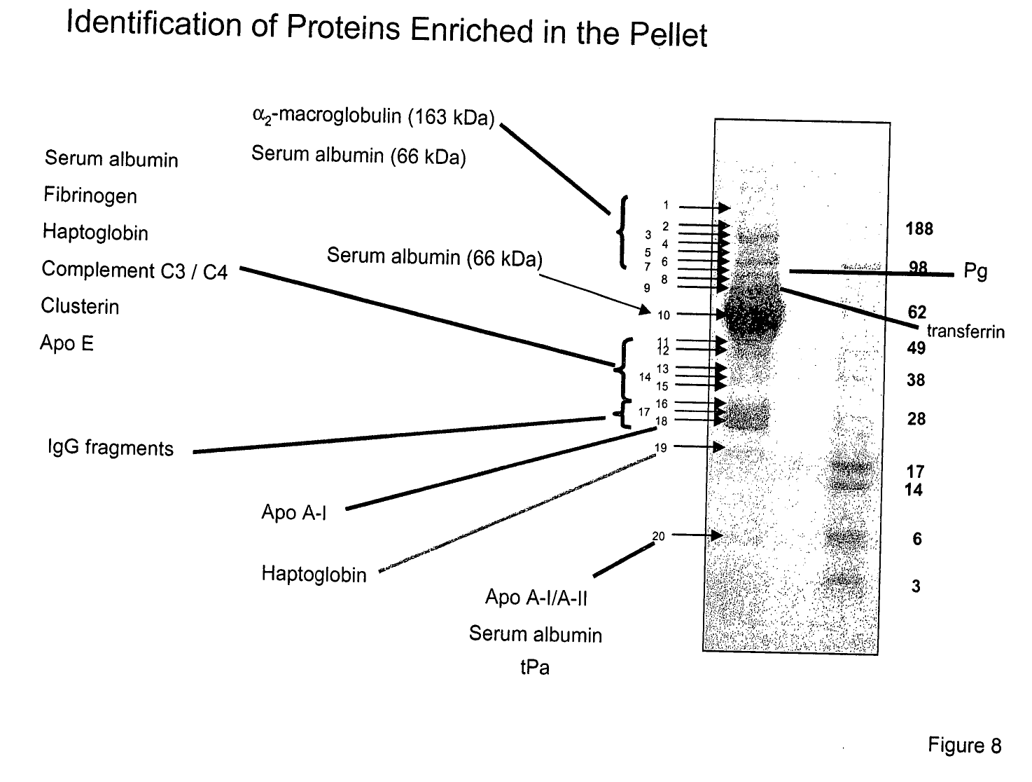

Identification of proteins enriched in the pellet phase formed using the reducing agent mediated precipitation methods according to the present invention as analyzed by in-gel tryptic digestion of gel bands are shown in FIG. 8. In addition to confirming the presence of transferrin and plasminogen (Pg) (˜75-90 kDa), the bands from approximately 98-188 kDa were highly albumin-rich, suggesting the presence of SDS-stable aggregates. In the 28-49 kDa MW range, proteins included albumin fragments, fibrinogen, haptoglobin, complement C3/C4, clusterin and ApoE. Overall, the darkest staining was observed for disulfide-linked proteins, albumin, α2-macroglobulin, IgG and transferrin.

### EXAMPLE IV

### Concentration Dependence of Albumin and Reducing Agent on Rate and Extent of Precipitation

The concentration dependence of albumin and reducing agent on the rate and extent of precipitation was explored. Plasma (in its natural state) was 2-fold serially diluted and treated with either reducing agent or PBS (control). In addition, the reducing agent concentrations were varied over final concentration ranges of 5-50 mM DTT and TCEP. The results are Table 1 below.

TABLE 1
The Effect of Reducing Agent and Plasma Concentrations on Albumin
Precipitation Rate

| Concentration of | | | | | |
|---|---|---|---|---|---|
| Reducing Agent (mM) | | | Plasma Dilution | | |
| | Initial^{@} | Final^{%} | Neat* | 2-fold | 4-fold |
| TCEP | 500 | 50 | 15 min | 70 min | ND^{#} |
| | 250 | 25 | 30 min | 60 min | ND^{#} |
| | 100 | 10 | 4 hours | 4 hours | ND^{#} |
| | 50 | 5 | >8 hours | >8 hours | >8 hours |
| DTT | 500 | 50 | 30 min | 60 min | 4 hours |
| | 250 | 25 | 1 hour | 3 hour | >4 hour |
| | 100 | 10 | 4 hour | 4 hour | >8 hour |
| | 50 | 5 | >8 hour | >8 hour | >8 hour |
| PBS | 0 | | — | — | — |
| control | | | | | |

^{@}Initial concentration refers to the stock concentration of the reducing agent
^{%}Final concentration refers to the concentration of the reducing agent in the total volume of specimen and reducing agent mixture.
*Neat refers to 9 parts biological specimen and 1 part reducing agent
^{#ND refers to no detectable precipitate after overnight incubation}

Table 1 illustrates several notable trends. Reaction rates clearly depend on reducing agent concentration or the concentration of the biomolecule to be removed (e.g., concentrated or depleted) from the biological specimen. When the reducing agent concentration or the biomolecule concentration is decreased, the time to form a precipitate increases.

These qualitative results also suggest that for precipitation reactions to be complete in reasonable timeframe (e.g., preferably less than 1 hour, more preferably <30 min), the plasma sample should be minimally diluted (preferably <2-fold) and TCEP is preferable over DTT because its higher reducing potential allows shorter times to precipitation compared to DTT at the same final concentration (i.e., concentration of reducing agent in total volume of specimen and reducing agent mixture).

In contrast, the non-incubation approach involves sample evaporation on a MALDI-MS target and therefore dilute plasma samples (20-fold in PBS) in the presence of reducing agents still exhibit minimal albumin ionization due to the sample drying/concentrating effect.

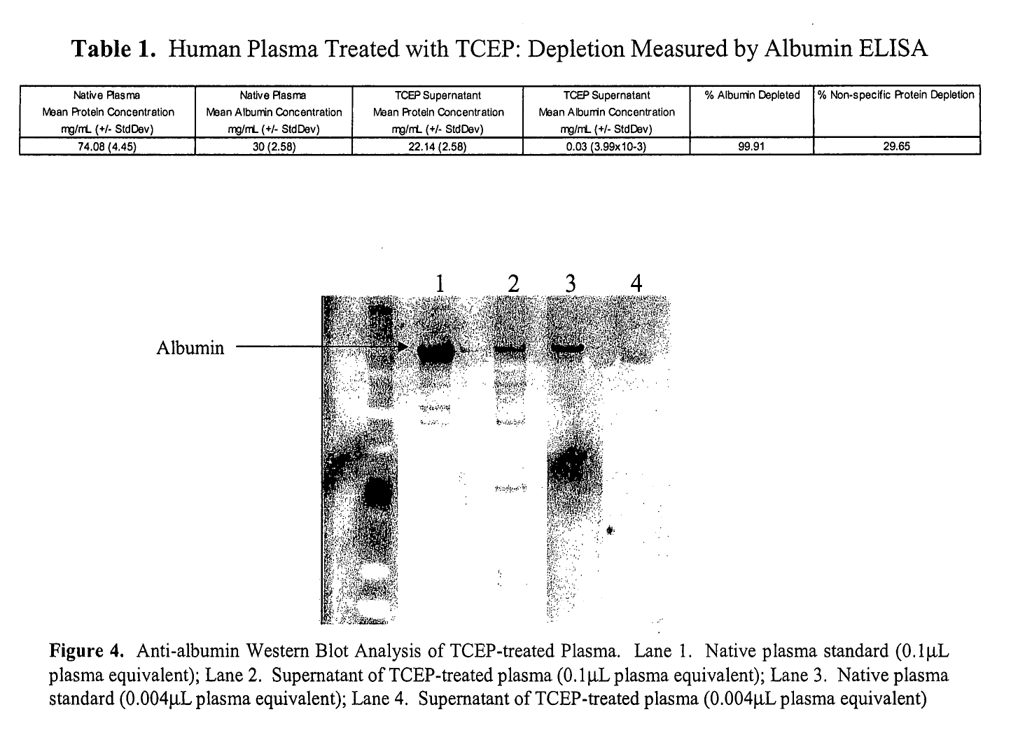

The degree of albumin depletion is comparable to values quoted from other commercially available albumin depletion kits that include dye resins and antibody-based beads (see Table 2 below and FIG. 4).

TABLE 2
Depletion of Albumin from Human Serum by Reducing Agent
Treatment and Commercially Available Kits

| | % Albumin | Other Protein |
|---|---|---|
| Depletion Method | Depletion1 | % Depleted |
| Amersham | 96.05 | 23.69 |
| Agilent Immunoaffinity | 99.2 | 15.09 |
| Spin Column | | |
| Pierce Swell Gel | 98.4 | 26.91 |
| Reducing Agent (TCEP) | 99.78 | 24.49 |

1Albumin depletion was measured by an anti-human ELISA
²This kit is designed to remove albumin and IgG
³The column has antibodies that bind to albumin, IgG, IgA, haptoglobin, transferrin and α-1-antitrypsin
⁴This kit is designed to deplete albumin (immobilized Cibracon Blue dye)

### EXAMPLE V

### Plasma Profiles from a Preclinical Mouse Model: Discovery of Treatment-Dependent Changes for Inhibitors of the Cancer Target Methionine Aminopeptidase (MetAP2)

The following chemicals and reagents were utilized in the method:

Tris(2-carboxyethyl)phosphine hydrochloride (TCEP), Sinapinic Acid, Dithiothreitol (DTT), and Trifluoroacetic acid were obtained from Sigma-Aldrich (St. Louis, Mo.). Acetonitrile was obtained from EMD Chemicals Inc (Gibbstown, N.J.). All water was distilled and deionized (resistivity >18.0 Megohm.cm). Female C57BL/6 mice (approximately 7 weeks old) were inoculated subcutaneously in the right flank with 0.2 mL of 6×10⁴ B16F10 cells (1:1 matrigel) on study day 0. Treatments were started on day 1 and continued until day 14. The mice were orally dosed twice a day, either with vehicle (carrier solution for the drug) or the covalent MetAP2 inhibitor, TNP-470. In this particular case study, there were 8 vehicle and 10 TNP-470 treated animals. At the end of the treatment the mice were terminally bled with heparin-charged syringes into potassium EDTA anticoagulant tubes. The tubes were centrifuged (1500×g, 10 min) to generate plasma and aliquots were frozen and stored at −80° C.

Reducing agent (TCEP, 250 mM in ddH₂O) was freshly prepared. Freshly thawed plasma (9 μL) was mixed with reducing agent (1 μL) and vortexed briefly. In this study, the plasma/reducing agent mixture was incubated until a viscous white precipitation persisted (generally 30-60 min). The sample was centrifuged (16-20,000×g, 10 min) prior to spotting the supernatant (1.0 μL) on a MALDI target.

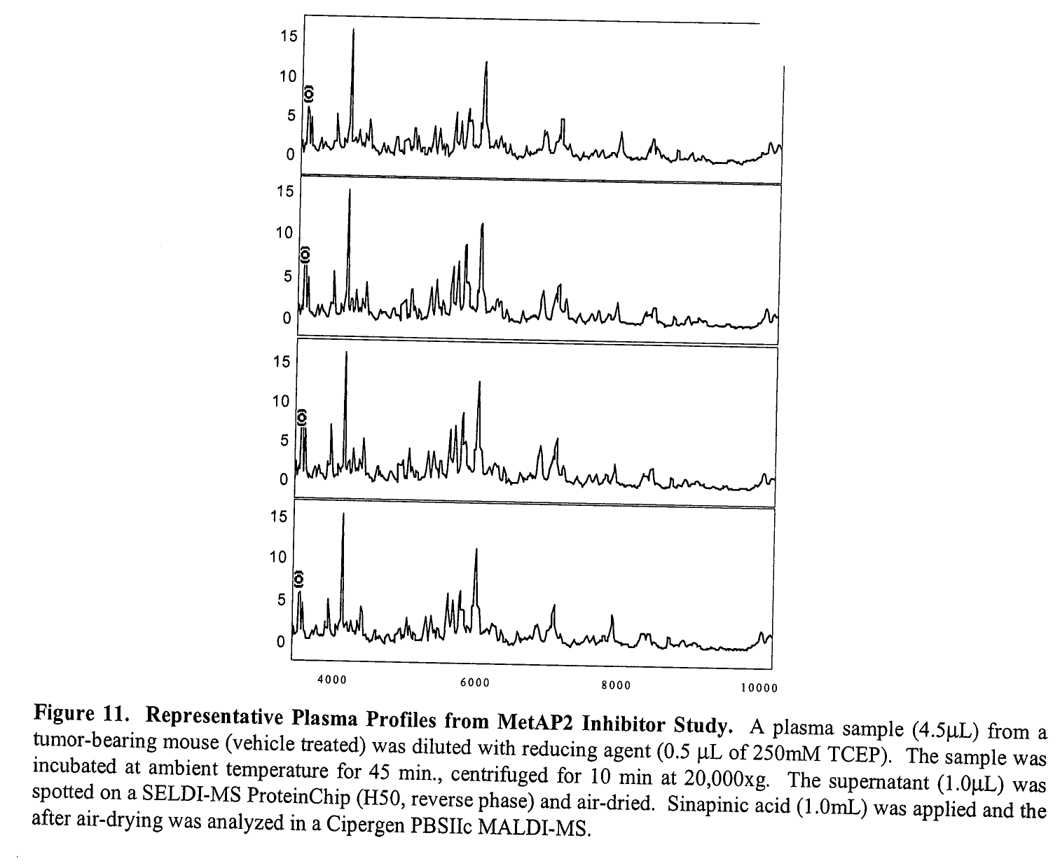

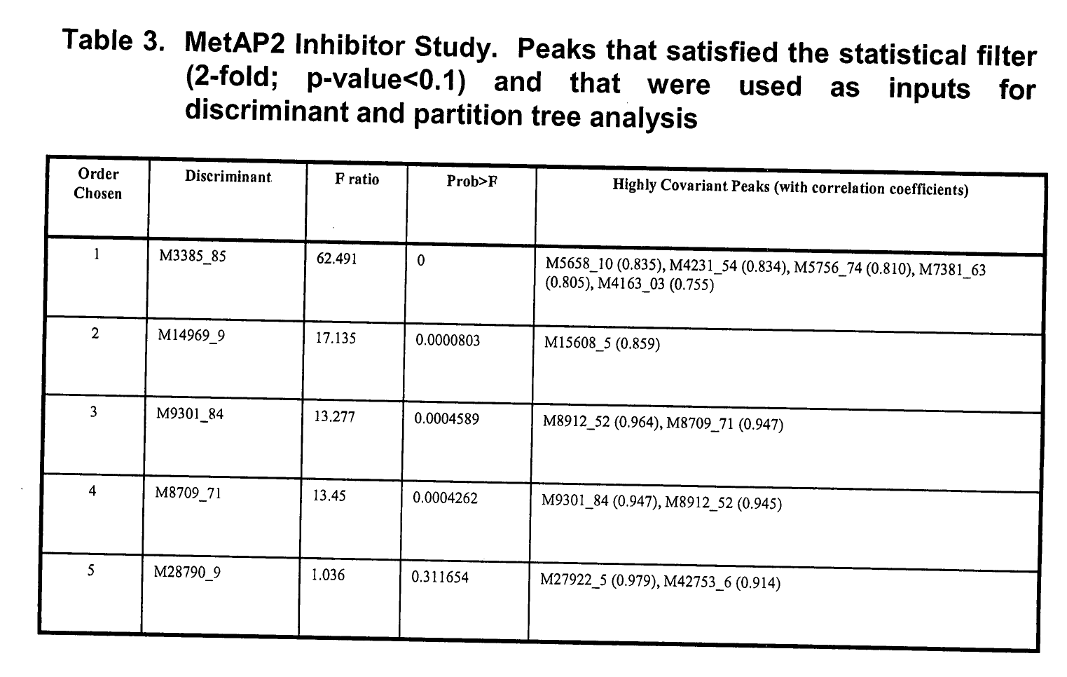

Data were collected on a Ciphergen Biosystems PBS-IIc (Fremont, Calif.) SELDI-TOF mass spectrometer. Mass spectra were recorded in linear positive ion mode at source voltage of 20 kV and a 1 GHz digitizer rate. Each spot was sampled using the same protocol: position 30 through 70 (0-100 scale), with 2 warming laser shots (not included in the average) and 8 laser shots (included in the average). The laser intensity was fixed at 220 (0-300 scale) and the detector sensitivity at 6 (0-10 scale). The PBSIIc TOF MS was externally calibrated using the “All-In-One” peptide mass standard from Ciphergen Biosystems, Inc. (Fremont, Calif.). The data were processed using the Ciphergen ProteinChip software. The baselines were smoothed using the default parameters. All spectra were normalized to each other using the ion current between 2500 m/z and 50,000 m/z. Representative traces are presented in FIG. 11. Peaks were detected and clustered using Ciphergen's Biomarker Wizard software package. Typically, peaks with s/n >10 were detected in the first pass and peaks with a s/n >5, included in the second pass. Peak inclusion was limited to peaks detected in at least 20% of all the spectra for a particular group. Peak lists and intensity information were exported and analyzed in an in-house program, SELDI Filter. Peaks fulfilling the criteria (e.g., 2-fold difference and p-value<0.1) are presented in Table 3 below.

TABLE 3

| Order | | | | Highly Covariant Peaks |
|---|---|---|---|---|
| Chosen | Discriminant | F ratio | Prob > F | (with correlation coefficients) |
| 1 | M3385_85 | 62.491 | 0 | M5658_10 (0.835), M4231_54 (0.834), |
| | | | | M5756_74 (0.810), M7381_63 (0.805), |
| | | | | M4163 03 (0.755) |
| 2 | M14969_9 | 17.135 | 0.0000803 | M15608_5 (0.859) |
| 3 | M9301_84 | 13.277 | 0.0004589 | M8912_52 (0.964), M8709_71 (0.947) |
| 4 | M8709_71 | 13.45 | 0.0004262 | M9301_84 (0.947), M8912_52 (0.945) |
| 5 | M28790_9 | 1.036 | 0.311654 | M27922_5 (0.979), M42753_6 (0.914) |

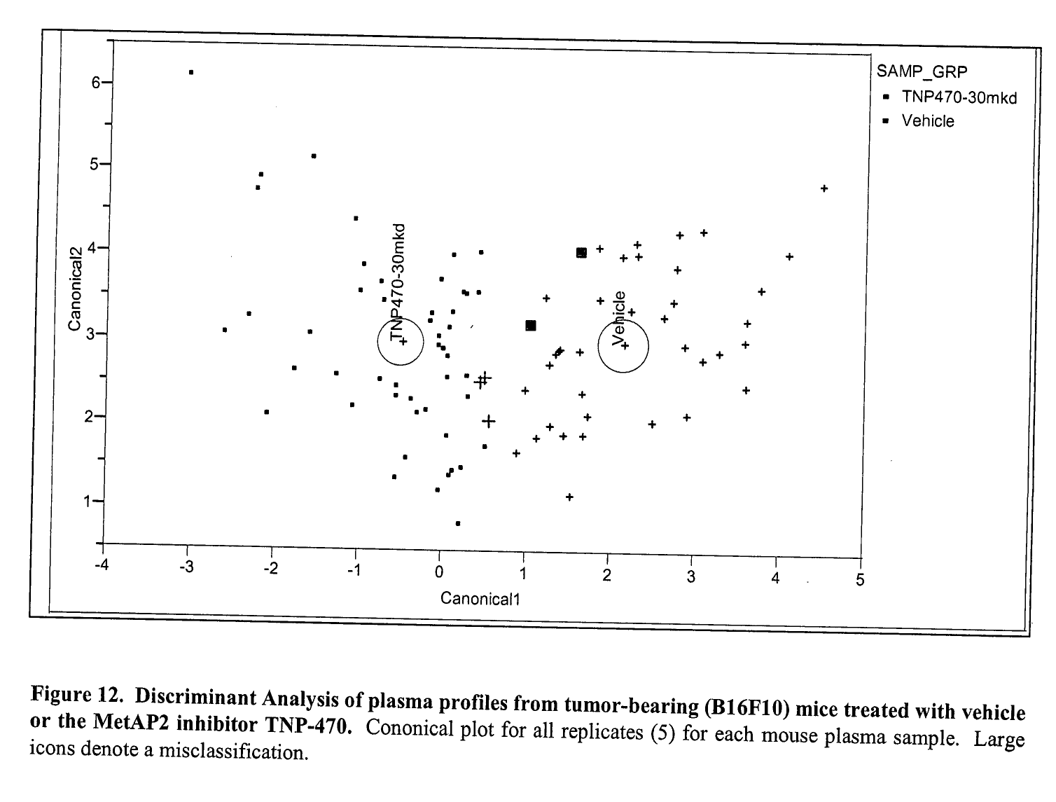

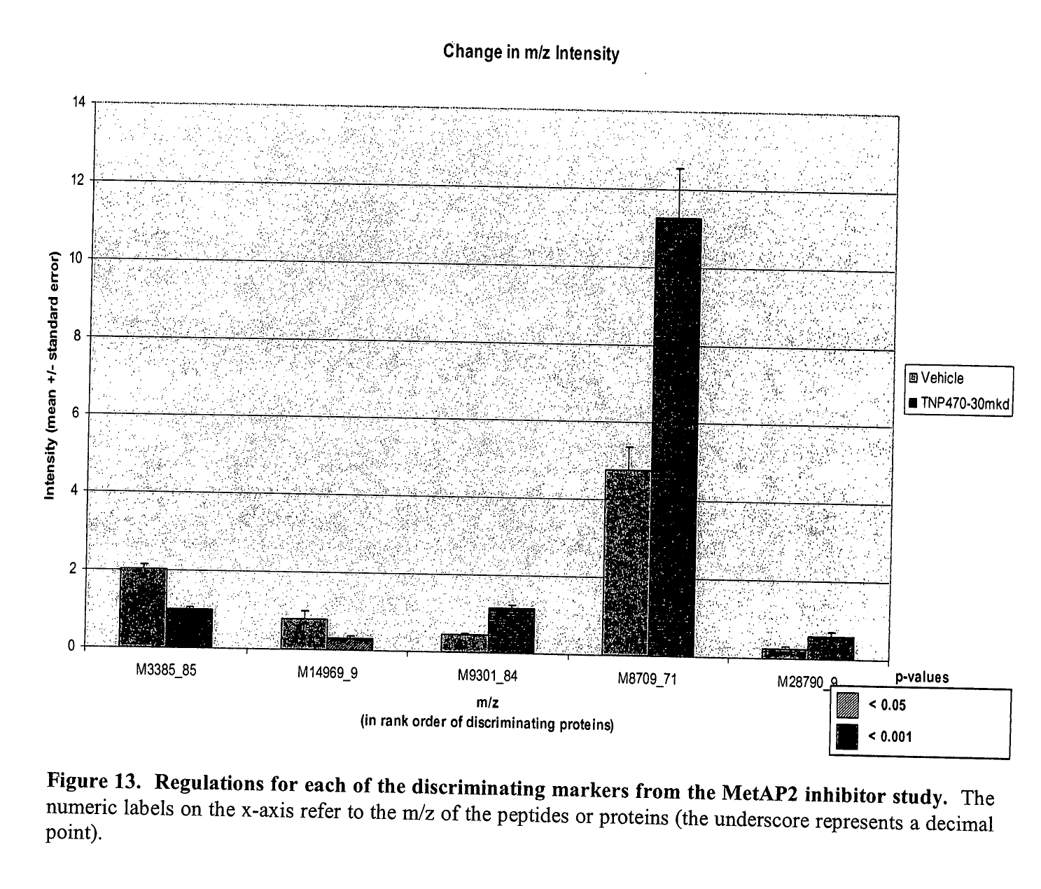

These peak intensities were directly inputted into the software package JMP for further analyses (e.g., discriminant analysis, partition tree, neural networks, etc.). An example of the discriminating power of these markers is presented in FIG. 12. The mean regulations with standard errors for all animals tested are presented in FIG. 13.

What is claimed is:

1. A method for separating a first biomolecule in a biological specimen from a second biomolecule in the biological specimen, wherein the second biomolecule has a relatively lower concentration in the biological specimen than the first biomolecule, comprising the steps of:
a. reacting the biological specimen with a reducing agent to create a first phase enriched in the first biomolecule and a second phase enriched in the second biomolecule, wherein the concentration of the first biomolecule in the biological specimen is sufficiently high to induce a precipitation; and
b. separating the first phase from the second phase.

2. A method according to claim 1, wherein the first phase is a solid.

3. A method according to claim 1, wherein the second phase is a liquid.

4. A method according to claim 1, wherein the first phase is a liquid.

5. A method according to claim 1, wherein the second phase is a solid.

6. A method according to claim 2, wherein the solid contains the second biomolecule.

7. A method according to claim 6, wherein the second biomolecule is associated with the first biomolecule.

8. A method according to claim 7, wherein the second biomolecule is associated with the first biomolecule by adsorption.

9. A method according to claim 7, wherein the second biomolecule is entangled with the first biomolecule.

10. A method according to claim 1, wherein the solid is created in step a. in less than one hour after mixing the reducing agent and the biological specimen.

11. A method according to claim 1, wherein the first biomolecule is a protein having disulfide-linked cysteines.

12. A method according to claim 11, wherein the reducing agent is selected from the group consisting of tris(2-carboxyethyl)phosphine (TCEP), dithiothreitol (DTT) and β-mercaptoethanol.

13. A method according to claim 1, wherein the biological specimen is selected from the group consisting of serum, whole blood, plasma, urine, cerebral spinal fluid, tears, semen, aqueous humor and intestinal fluid.

14. A method according to claim 1, wherein the biological specimen is a formulated blood component-containing specimen.

15. A method according to claim 14, wherein the formulated blood component-containing specimen is selected from the group consisting of tissue culture supernatant, cell culture supernatant, and samples stabilized with serum components.

16. A method according to claim 1, wherein the biological specimen is from a mammal.

17. A method according to claim 1, wherein the concentration of reducing agent is sufficiently high to create the first phase and the second phase.

18. A method for discovering a biomarker associated with a biological state of interest, comprising the steps of:
a. providing a plurality of biological specimens, each specimen representing either a presence or absence of the biological state of interest and being minimally diluted from their respective natural states;
b. treating each of the plurality of biological specimens with a reducing agent to form a precipitate from each specimen, wherein the precipitate contains at least one biomolecule having a disulfide-linked cysteine bond;
c. analyzing each treated specimen to provide a profile of biomarker content in each specimen;
d. identifying a biomarker that is differentially regulated as a result of the different biological states.

19. A method according to claim 18, wherein the precipitate is created in step b. in less than one hour after mixing the reducing agent and the biological specimen.

20. A method according to claim 18, wherein the first biomolecule is a protein having disulfide-linked cysteines.

21. A method according to claim 18, wherein the reducing agent is selected from the group consisting of tris(2-carboxyethyl)phosphine (TCEP), dithiothreitol (DTT) and β-mercaptoethanol.

22. A method according to claim 18, wherein the biological specimen is selected from the group consisting of serum, whole blood, plasma, urine, cerebral spinal fluid, tears, semen, aqueous humor and intestinal fluid.

23. A method according to claim 18, wherein the biological specimen is a formulated blood component-containing specimen.

24. A method according to claim 18, wherein the formulated blood component-containing specimen is selected from the group consisting of tissue culture supernatant, cell culture supernatant, and samples stabilized with serum components.

25. A method for depleting a first biomolecule in a biological specimen to allow monitoring of a second biomolecule in the biological specimen, comprising the steps of:
a) reacting the biological specimen with a reducing agent to create a first phase enriched in the first biomolecule and a second phase enriched in the second biomolecule, wherein the concentration of the first biomolecule in the biological specimen is sufficiently high to induce a precipitation; and
b) separating the first phase from the second phase.

26. A method according to claim 25, wherein the first phase is a solid.

27. A method according to claim 25, wherein the second phase is a liquid.

28. A method according to claim 25, wherein the first phase is a liquid.

29. A method according to claim 25, wherein the second phase is a solid.

30. A method according to claim 26, wherein the solid contains the second biomolecule.

31. A method according to claim 30, wherein the second biomolecule is associated with the first biomolecule.

32. A method according to claim 31, wherein the second biomolecule is associated with the first biomolecule by adsorption.

33. A method according to claim 32, wherein the second biomolecule is entangled with the first biomolecule.

34. A method according to claim 25, wherein the solid is created in step a. in less than one hour after mixing the reducing agent and the biological specimen.

35. A method according to claim 25, wherein the first biomolecule is a protein having disulfide-linked cysteines.

36. A method according to claim 35, wherein the reducing agent is selected from the group consisting of tris(2-carboxyethyl)phosphine (TCEP), dithiothreitol (DTT) and β-mercaptoethanol.

37. A method according to claim 36, wherein the biological specimen is selected from the group consisting of serum, whole blood, plasma, urine, cerebral spinal fluid, tears, semen, aqueous humor and intestinal fluid.

38. A method according to claim 25, wherein the biological specimen is a formulated blood component-containing specimen.

39. A method according to claim 38, wherein the formulated blood component-containing specimen is selected from the group consisting of tissue culture supernatant, cell culture supernatant, and samples stabilized with serum components.

40. A method according to claim 25, wherein the biological specimen is from a mammal.

41. A method according to claim 25, wherein the concentration of reducing agent is sufficiently high to create the first phase and the second phase.

42. A method of reducing the amount of albumin contained in a sample of a biological fluid comprising the steps of:
a) mixing said sample with a reducing agent;
b) depositing said mixed sample on a target;
c) drying said sample;
d) applying a matrix;
e) drying said matrix;
f) applying a source of photo-excitation to said dried matrix of step e); and
g) profiling said sample in order to ensure reduction of said amount of albumin contained in said sample.

43. The method of claim 42 wherein said biological fluid is selected from the group consisting of serum, whole blood, plasma, urine and cerebral spinal fluid.

44. The method of claim 42 wherein said reducing agent is selected from the group consisting of tris(2-carboxyethyl)phosphine (TCEP), dithiothreitol (DTT) and β-mercaptoethanol.

45. The method of claim 44 wherein said reducing agent is used at a final concentration of at least about 5 mm.

46. The amount of claim 44 wherein said reducing agent is used at a final concentration of at most about 50 mM.

47. The method of claim 42 wherein said target is a Matrix-Assisted Laser Desorption Ionization Time-Of-Flight Mass Spectrometry (MALDI-TOF-MS) sample introduction device or a Surface-Enhanced Laser Desorption Ionization Time-Of-Flight Mass Spectrometry (SELDI-TOF-MS) sample introduction device.

48. The method of claim 42 wherein drying step c) is accomplished through application of a source selected from the group consisting of vacuum, air and heat.

49. The method of claim 42 wherein said profiling is accomplished by use of a Matrix-Assisted Laser Desorption Ionization Time-Of-Flight Mass Spectrometry (MALDI-TOF-MS) instrument.

50. The method of claim 42 wherein said biological fluid is from a mammal.

51. The method of claim 50 wherein said biological fluid comprises at least one biomolecule selected from the group consisting of a peptide, a protein, a nucleic acid and a small organic or inorganic molecule which may ionize in a mass spectrometer.

52. The method of claim 42 wherein said matrix is selected from the group consisting of alpha-cyano-4-hydroxycinnamic acid (CHCA), sinapinic acid (SPA) and 2,5-dihydroxy benzoic Acid (DHB).

53. The method of claim 42 wherein photoexcitation of said matrix results in protonation or deprotonation of biomolecules in said biological fluid sample.

54. A method of reducing the amount of albumin contained in a sample of a biological fluid comprising the steps of:
a. mixing said sample with a reducing agent;
b. incubating said mixed sample until formation of a precipitate occurs;
c. centrifuging said incubated sample;
d. recovering supernatant from said resulting centrifuged sample;
e. depositing said centrifuged sample on a target;
f. drying said centrifuged sample;
g. applying a matrix to said centrifuged sample;
h. drying said matrix;
i. applying a source of photo-excitation to said dried matrix; and
j. profiling said centrifuged sample in order to ensure reduction of amount of albumin contained in said sample.

55. The method of claim 54 wherein said biological fluid is selected from the group consisting of serum, whole blood, plasma, urine and cerebral spinal fluid (CSF).

56. The method of claim 54 wherein said reducing agent is selected from the group consisting of tris(2-carboxyethyl)phosphine hydrochloride (TCEP), dithiothreitol (DTT) and β-mercaptoethanol.

57. The method of claim 54 wherein said reducing agent is used in a final concentration of at least about 5 mm.

58. The amount of claim 54 wherein said reducing agent is used in a final concentration of at most about 50 mM.

59. The method of claim 54 wherein said target is a MALDI-TOF-MS or SELDI-TOF-MS sample introduction device.

60. The method of claim 54 wherein drying step f) is accomplished through application of a source selected from the group consisting of vacuum, air and heat.

61. The method of claim 54 wherein said profiling is accomplished by use of a MALDI-TOF-MS instrument.

62. The method of claim 54 wherein said biological fluid is from a mammal.

63. The method of claim 62 wherein said biological fluid comprises at least one biomolecule selected from the group consisting of a peptide, a protein, a nucleic acid and a small organic or inorganic molecule which may ionize in a mass spectrometer.

64. The method of claim 54 wherein said matrix is selected from the group consisting of alpha-cyano-4-hydroxycinnamic acid (CHCA), sinapinic acid (SPA) and 2,5-dihydroxy benzoic acid (DHB).

65. The method of claim 54 wherein photoexcitation of said matrix results in protonation or deprotonation of biomolecules in said biological fluid sample.
